(19) United States
(12) Patent Application Publication
(10) Pub. No.: US 2015/0375196 A1
(43) Pub. Date: Dec. 31, 2015

(54) PROCESS FOR CONVERSION OF PROPANE AND APPARATUS

(71) Applicant: UOP LLC, Des Plaines, IL (US)

(72) Inventors: Christopher P. Nicholas, Evanston, IL (US); Charles P. Luebke, Mount Prospect, IL (US); Steven L. Krupa, Fox River Grove, IL (US); Charles M. Brabson, Humble, TX (US)

(21) Appl. No.: 14/752,028

(22) Filed: Jun. 26, 2015

Related U.S. Application Data
(60) Provisional application No. 62/018,933, filed on Jun. 30, 2014.

(51) Int. Cl. B01J 19/24; C07C 2/12; C07C 5/327

(52) U.S. Cl. CPC B01J 19/245; C07C 5/327; C07C 2/12; B01J 2219/24; C07C 2529/06

(57) ABSTRACT

A process and apparatus are presented for the conversion of propane to higher valued fuels, such as gasoline and diesel. The process includes the dehydrogenation of propane to generate a propylene stream. The propylene stream is oligomerized and controlled to generate a liquid hydrocarbon stream in the C6 to C12 range.

### CROSS REFERENCE TO RELATED APPLICATION

This application claims the benefit of U.S. Provisional Application No. 62/018,933 which was filed on Jun. 30, 2014 , the contents of which are hereby incorporated by reference in its entirety.

### FIELD OF THE INVENTION

The field of the invention pertains to the production of liquid fuels from light hydrocarbon feedstocks. In particular, the process is for the conversion of propane into fuels such as gasoline and diesel.

### BACKGROUND

The oligomerization of olefins is known. Oligomerization is carried out by reacting olefinic hydrocarbons over catalysts to obtain various oligomers. Oligomerization is often applied to the process of forming dimers, trimers, and tetramers from monomers, and also can cover polymerization processes.

One oligomerization process is described in U.S. Pat. No. 3,697,617 and involves the use of a catalyst that comprises a complex of nickel and an electron donor ligand. The complex is deposited on a solid support of an acidic material such as silica-alumina. The polymerization of an olefin is described in U.S. Pat. No. 3,644,564, using a nickel compound catalyst. U.S. Pat. No. 3,644,564 describes the oligomerization of ethylene. Other patents such as U.S. Pat. No. 8,178,740 describe catalysts for the oligomerization or polymerization of olefins.

When oligomerizing light olefins within a refinery, there is frequently a desire to have the flexibility to make high octane gasoline, high cetane diesel, or combination of both. However, catalysts that make high octane gasoline typically make product that is highly branched and within the gasoline boiling point range. This product is very undesirable for diesel. In addition, catalysts that make high cetane diesel typically make product that is more linear and in the distillate boiling point range. This results in less and poorer quality gasoline due to the more linear nature of the product which has a lower octane value.

To maximize distillate produced in a refinery, refiners may contemplate oligomerizing FCC derived light olefins to make heavier oligomers, thereby shifting gasoline into the distillate range. However, not all refiners have cost advantaged FCC derived light olefin streams available. In some cases, light paraffins are the most cost advantaged feed.

Light paraffins are incapable of being converted to liquid fuels by oligomerization directly. Combination processes for the conversion of light alkanes to light alkenes, followed by oligomerization of the alkene to liquid fuels are known. U.S. Pat. No. 4,293,722 describes a process comprising dehydrogenation of propane followed by catalytic condensation to form C9 hydrocarbons, U.S. Pat. No. 4,304,948 describes a process comprising dehydrogenation of butane followed by catalytic condensation to form C8-C12 hydrocarbons, U.S. Pat. No. 4,542,247 describes a process comprising dehydrogenation of paraffins followed by 2 steps of oligomerization with an intermediate separation step, U.S. Pat. No. 6,897,345 describes a process comprising the isomerization of n-butane to isobutane followed by dehydrogenation and dimerization, U.S. Pat. No. 5,856,604 describes a process comprising dehydrogenation of isobutane, compression and oligomerization, U.S. Pat. No. 5,847,252 describes a process comprising low severity dehydrogenation of isobutane, oligomerization and saturation and U.S. Pat. No. 4,879,424 describes a process comprising feeding a heated feedstream to a zeolite catalyst to form olefins and aromatics and then passing this product stream to a second reaction zone where oligomerization occurs. These combined processes suffer from drawbacks such as low pressure oligomerization, aromatic formation or formation of high quantities of gasoline. Distillate range products are desired, particularly with a high cetane number.

While there are catalysts and processes for the oligomerization of olefins and for the conversion of propane to liquid fuels, there is a need for improved processes and increased products, particularly distillate, that result from oligomerization of olefins.

### SUMMARY

A first embodiment of the invention is an apparatus for the production of distillate, comprising a dehydrogenation zone having an inlet and an outlet for passage of an olefin enriched stream; a deethanizer unit having an inlet in fluid communication with the dehydrogenation zone outlet, an overhead outlet for light ends, and a bottoms outlet for C3s; a first oligomerization zone having an inlet in fluid communication with the dehydrogenation zone outlet, and an outlet for the passage of a hydrocarbon stream comprising C6 to C12 hydrocarbons; a first fractionation unit having an inlet in fluid communication with the first oligomerization zone outlet, and a first fractionation unit overhead outlet for the passage of C3s, and a first fractionation unit bottoms outlet for the passage of C₆+ hydrocarbons; a second oligomerization zone having an inlet in fluid communication with the first fractionation unit bottoms outlet, and an outlet; and a second fractionation unit having an inlet in fluid communication with the second oligomerization zone outlet, a second fractionation unit overhead outlet for the passage of an olefinic process stream, and a second fractionation unit bottoms outlet for the passage of a distillate process stream.

A second embodiment of the invention is an apparatus for the production of distillate, comprising a dehydrogenation unit having an inlet and an outlet for passage of an olefin enriched stream; a deethanizer unit having an inlet in fluid communication with the dehydrogenation unit outlet, an overhead outlet for light ends, and a bottoms outlet for C3s; an aromatics removal unit having an inlet in fluid communication with the deethanizer bottoms outlet, and an outlet for a C3 stream with reduced aromatics; a first oligomerization zone having an inlet in fluid communication with the aromatics removal unit outlet, and an outlet for the passage of a hydrocarbon stream comprising C6 to C12 hydrocarbons; a first fractionation unit having an inlet in fluid communication with the first oligomerization zone outlet, and a first fractionation unit overhead outlet for the passage of C3s, and a first fractionation unit bottoms outlet for the passage of C6 to C12 hydrocarbons; a second oligomerization zone having an inlet in fluid communication with the first fractionation unit bottoms outlet, and an outlet; a second fractionation unit having an inlet in fluid communication with the second oligomerization zone outlet, a second fractionation unit overhead outlet for the passage of an olefinic process stream, and a second fractionation unit bottoms outlet for the passage of a distillate process stream; and a first conduit in direct communication with the outlet of the first fractionation overhead and the inlet to the dehydrogenation unit.

Other objects, advantages and applications of the present invention will become apparent to those skilled in the art from the following detailed description and drawings.

### BRIEF DESCRIPTION OF THE DRAWINGS

FIG. 1 is a schematic for a first embodiment of the process and description of the equipment for the oligomerization of paraffins; and

FIG. 2 is a schematic for a second embodiment of the process and description for the conversion of light paraffins to heavier hydrocarbons.

### DEFINITIONS

As used herein, the term “stream” can include various hydrocarbon molecules and other substances. Moreover, the term “stream comprising Cx hydrocarbons” or “stream comprising Cx olefins” can include a stream comprising hydrocarbon or olefin molecules, respectively, with “x” number of carbon atoms, suitably a stream with a majority of hydrocarbons or olefins, respectively, with “x” number of carbon atoms and preferably a stream with at least 75 wt % hydrocarbons or olefin molecules, respectively, with “x” number of carbon atoms. Moreover, the term “stream comprising Cx+ hydrocarbons” or “stream comprising Cx+ olefins” can include a stream comprising a majority of hydrocarbon or olefin molecules, respectively, with more than or equal to “x” carbon atoms and suitably less than 10 wt % and preferably less than 1 wt % hydrocarbon or olefin molecules, respectively, with x−1 carbon atoms. Lastly, the term “Cx− stream” can include a stream comprising a majority of hydrocarbon or olefin molecules, respectively, with less than or equal to “x” carbon atoms and suitably less than 10 wt % and preferably less than 1 wt % hydrocarbon or olefin molecules, respectively, with x+1 carbon atoms.

As used herein, the term “zone” can refer to an area including one or more equipment items and/or one or more sub-zones. Equipment items can include one or more reactors or reactor vessels, heaters, exchangers, pipes, pumps, compressors, controllers and columns. Additionally, an equipment item, such as a reactor, dryer, or vessel, can further include one or more zones or sub-zones.

As used herein, the term “substantially” can mean an amount of at least generally about 70%, preferably about 80%, and optimally about 90%, or more, by weight, of a compound or class of compounds in a stream.

As used herein, the term “gasoline” can include hydrocarbons having a boiling point temperature in the range of about 25° to about 200° C. at atmospheric pressure.

As used herein, the term “diesel” or “distillate” can include hydrocarbons having a boiling point temperature in the range of about 150° to about 400° C. and preferably about 200° to about 400° C.

As used herein, the term “light paraffin” indicates streams comprising propane. Preferably, the propane substantially comprises the stream.

As used herein, the term “light olefin” indicates hydrocarbon streams comprising one or more C3-C5 olefins derived from dehydrogenation of light paraffins.

As used herein, the term “vapor” can mean a gas or a dispersion that may include or consist of one or more hydrocarbons.

As used herein, the term “overhead stream” can mean a stream withdrawn at or near a top of a vessel, such as a column.

As used herein, the term “bottom stream” can mean a stream withdrawn at or near a bottom of a vessel, such as a column.

As depicted, process flow lines in the figures can be referred to interchangeably as, e.g., lines, pipes, feeds, gases, products, discharges, parts, portions, or streams.

As used herein, “bypassing” with respect to a vessel or zone means that a stream does not pass through the zone or vessel bypassed although it may pass through a vessel or zone that is not designated as bypassed.

The term “communication” or “fluid communication” means that material flow is operatively permitted between enumerated components.

The term “downstream communication” means that at least a portion of material flowing to the subject in downstream communication may operatively flow from the object with which it communicates.

The term “upstream communication” means that at least a portion of the material flowing from the subject in upstream communication may operatively flow to the object with which it communicates.

The term “direct communication” means that flow from the upstream component enters the downstream component without undergoing a compositional change due to physical fractionation or chemical conversion.

The term “column” means a distillation column or columns for separating one or more components of different volatilities. Unless otherwise indicated, each column includes a condenser on an overhead of the column to condense and reflux a portion of an overhead stream back to the top of the column and a reboiler at a bottom of the column to vaporize and send a portion of a bottom stream back to the bottom of the column. Feeds to the columns may be preheated. The top pressure is the pressure of the overhead vapor at the outlet of the column. The bottom temperature is the liquid bottom outlet temperature. Overhead lines and bottom lines refer to the net lines from the column downstream of the reflux or reboil to the column.

As used herein, the term “boiling point temperature” means atmospheric equivalent boiling point (AEBP) as calculated from the observed boiling temperature and the distillation pressure, as calculated using the equations furnished in ASTM D1160 appendix A7 entitled “Practice for Converting Observed Vapor Temperatures to Atmospheric Equivalent Temperatures”.

As used herein, “taking a stream from” means that some or all of the original stream is taken.

### DETAILED DESCRIPTION

There is a continuing demand for liquid fuels, and there is a continuing growth in the recovery of light hydrocarbons from natural gas sources, and/or gas stream from oil producing sources. Light olefin oligomerization has used FCC process technology for the production of light olefins as feed. To expand the market, sources of materials include light paraffins. However, light paraffins cannot be fed directly to an oligomerization reactor as the unreactivity of paraffins leads to no reaction with paraffins in an oligomerization reactor.

When oligomerizing light olefins within a refinery, there is frequently a desire to have the flexibility to make high octane gasoline, high cetane diesel, or combination of both. However, catalysts that make high octane gasoline typically make product that is highly branched and within the gasoline boiling point range. This product is very undesirable for diesel. In addition, catalysts that make high cetane diesel typically make product that is more linear and in the distillate boiling point range. This results in less and poorer quality gasoline due to the more linear nature of the product which has a lower octane value.

When oligomerizing olefins, there is often the desire to maintain a liquid phase within the oligomerization reactors. A liquid phase helps with catalyst stability by acting as a solvent to wash the catalyst of heavier species produced. In addition, the liquid phase provides a higher concentration of olefins to the catalyst surface to achieve a higher catalyst activity. Typically, this liquid phase in the reactor is maintained by hydrogenating some of the heavy olefinic product and recycling this paraffinic product to the reactor inlet.

The products of olefin oligomerization are usually mixtures of, for example, olefin dimers, trimers, and higher oligomers. Further, each olefin oligomer is itself usually a mixture of isomers, both skeletal and in double bond location. Highly branched isomers are less reactive than linear or lightly branched materials in many of the downstream reactions for which oligomers are used as feedstocks. This is also true of isomers in which access to the double bond is sterically hindered. Olefin types of the oligomers can be denominated according to the degree of substitution of the double bond, as follows:

TABLE 1

| Olefin Type | Structure | Description |
|---|---|---|
| I | R—HC═CH₂ | Monosubstituted |
| II | R—HC═CH—R | Disubstituted |
| III | RRC═CH₂ | Disubstituted |
| IV | RRC═CHR | Trisubstituted |
| V | RRC═CRR | Tetrasubstituted |

wherein R represents an alkyl group, each R being the same or different. Type I compounds are sometimes described as α- or vinyl olefins and Type III as vinylidene olefins. Type IV is sometimes subdivided to provide a Type IVA, in which access to the double bond is less hindered, and Type IVB where it is more hindered.

The present invention is combining processes for taking lower valued light paraffins and converting them to more valuable liquid products, such as distillates. Distillates as used herein refer to hydrocarbon product streams that comprise C10+ hydrocarbons and which can be used, for example, as diesel fuel. The process includes the integration of the dehydrogenation of light alkanes with catalytic oligomerization with the integration of appropriate separation processes to maximize the production of a distillate product stream.

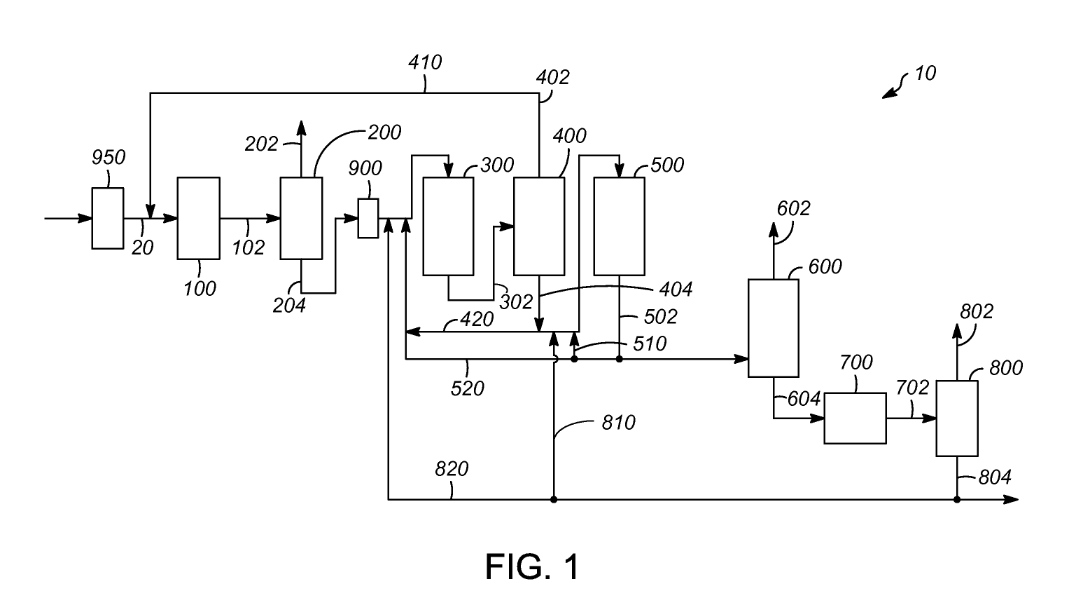

An apparatus is presented as shown in FIG. 1 for the production of distillate. The apparatus 10 includes a dehydrogenation zone 100 having an inlet for admitting a light paraffin stream 20 and an outlet for passing an olefin enriched stream 102. The apparatus 10 further includes a deethanizer 200 having an inlet in fluid communication with the dehydrogenation zone 100 outlet, an overhead outlet for a light ends stream 202 and a bottoms outlet for a stream comprising C3 hydrocarbons 204. The apparatus 10 also includes a first oligomerization zone 300 having an inlet in fluid communication with the deethanizer bottoms outlet, and an outlet for the passage of a hydrocarbon stream comprising C₆+ hydrocarbons and unreacted lighter hydrocarbons 302. The apparatus 10 includes a first fractionation unit 400 for separating the oligomerized products from the unreacted components, and where the fractionation unit includes an overhead outlet for the passage of a stream comprising propane 402, and a bottoms outlet for the passage of heavier hydrocarbons, such as a stream comprising C₆+ hydrocarbons 404. The apparatus 10 includes a second oligomerization zone 500 having an inlet in fluid communication with the first fractionation unit bottoms outlet and an outlet for passage of the oligomerized process stream, comprising distillate 502. The apparatus 10 further includes a second fractionation unit 600 having an inlet in fluid communication with the second oligomerization unit 500 outlet and a second fractionation unit overhead outlet for the passage of an isoolefinic process stream 602, and a second fractionation unit bottoms stream for the passage of a distillate process stream 604.

The distillate process stream can further be treated. The apparatus 10 can further include a first complete saturation process unit 700 having an inlet in fluid communication with the second fractionation unit 600 bottoms outlet and an outlet for the passage of a hydrogenated distillate process stream 702; and a product stripper 800 having an inlet in fluid communication with the complete saturation unit 700 outlet, a stripper overhead outlet for light ends 802, and a stripper bottoms outlet for distillate product 804.

The apparatus 10 can include conduits for recycle and passage of various streams to other zones or units within the apparatus. The apparatus 10 can optionally include a first conduit 510 having an inlet connecting a portion of the second oligomerization zone outlet, and an outlet in fluid communication with the second oligomerization zone inlet. The apparatus 10 can further include a second conduit 410 having an inlet connecting a portion of the first fractionation unit 400 overhead outlet, and an outlet in fluid communication with the inlet of the dehydrogenation unit 100. The apparatus 10 can include additional conduits for recycle, and a third conduit 420 provides an inlet in fluid communication with of the first fractionation unit bottoms stream, and an outlet in fluid communication with the inlet to the first oligomerization zone 300. The apparatus 10 can include a fourth conduit 810 having an inlet in fluid communication with the product stripper 800 bottoms outlet, and an inlet in fluid communication with the inlet to the second oligomerization zone 500. The apparatus can include a fifth conduit 520 having an inlet connecting a portion of the second oligomerization zone 500 outlet, and an outlet in fluid communication with the first oligomerization zone 300 inlet. The apparatus can include a sixth conduit 820 having an inlet in fluid communication with the product stripper 800 bottoms outlet, and an outlet in fluid communication with the inlet to the first oligomerization zone 300.

The apparatus 10 can also include an aromatics removal unit 900 having an inlet in fluid communication with the deethanizer bottoms outlet, and an outlet in fluid communication with the first oligomerization zone 300 inlet.

The apparatus 10 can also include a contaminants removal zone 950 having an inlet to receive the light paraffin stream, and an outlet in fluid communication with the dehydrogenation zone 100.

The first oligomerization zone 300, or the second oligomerization zone 500, or both can include a plurality of reactors in a series arrangement. The oligomerization zones, 300, 500 include a catalyst for carrying out the oligomerization reactions. Oligomerization catalysts include zeolites having TON, MTT, MFI, MEL, AFO, AEL, EUO and FER type structures, solid phosphoric acid catalysts, and mixtures of the different zeolite structures. The catalysts can also include a support and a binder.

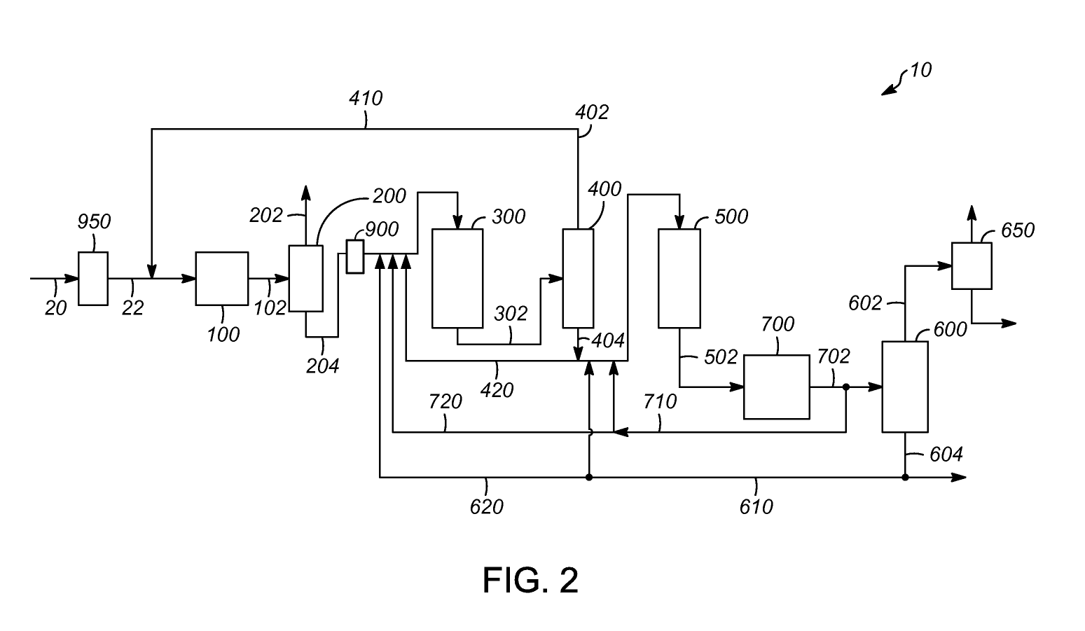

In a second embodiment of an apparatus for the present invention is shown in FIG. 2. The apparatus 10 includes a includes a dehydrogenation zone 100 having an inlet for admitting a light paraffin stream and an outlet for passing an olefin enriched stream. The dehydrogenation zone 100 is for propane dehydrogenation to yield a process stream comprising propylene. The apparatus 10 further includes a deethanizer 200 having an inlet in fluid communication with the dehydrogenation zone 100 outlet, an overhead outlet for light ends and a bottoms outlet for C3 and heavier hydrocarbons. The apparatus 10 includes an aromatics removal unit 900 having an inlet in fluid communication with the deethanizer bottoms outlet, and an outlet comprising an olefin stream having a reduced aromatics content. The apparatus 10 also includes a first oligomerization zone 300 having an inlet in fluid communication with the aromatics removal unit outlet, and an outlet for the passage of a hydrocarbon stream comprising C₆+ hydrocarbons and unreacted lighter hydrocarbons. The apparatus 10 includes a first fractionation unit 400 for separating the oligomerized products from the unreacted components, and where the fractionation unit includes an overhead outlet for the passage of unreacted C3s 402, and a bottoms outlet for the passage of heavier hydrocarbons 404. The apparatus 10 includes a second oligomerization zone 500 having an inlet in fluid communication with the first fractionation unit 400 bottoms outlet and an outlet for passage of the oligomerized process stream.

The apparatus 10 further includes a second fractionation unit 600 having an inlet in fluid communication with the second oligomerization unit 500 outlet. In this embodiment, the apparatus 10 can include a complete saturation unit 700 having an inlet in fluid communication with the second oligomerization zone 500 outlet, and an outlet in fluid communication with the inlet to the second fractionation unit 600, through which the second oligomerization process stream passes to reach the second fractionation unit 600.

The apparatus 10 includes a first conduit 410 having a direct fluid communication between the first fractionation unit 400 outlet and the inlet to the dehydrogenation unit 100.

In this embodiment, the second fractionation unit 600 can generate a bottoms stream of a distillate product 604, and an overhead stream 602. The overhead stream can be passed to an overhead stripping unit 650 to remove light hydrocarbons generated in the complete saturation unit 700.

The apparatus 10 can further include a second conduit 710 having an inlet in fluid communication with the complete saturation process unit outlet, and an inlet in fluid communication with the second oligomerization zone inlet. There can be a further conduit 720 having an inlet in fluid communication with the complete saturation process unit outlet, and an inlet in fluid communication with the first oligomerization zone inlet.

The piping can allow for a conduit 420 to connect the first fractionation zone 400 bottoms stream to the first oligomerization zone 300 inlet. An additional conduit 610 can connect the second fractionation zone 600 bottoms outlet with the second oligomerization zone 500 inlet, and an additional conduit 620 can connect the second fractionation zone 600 bottoms outlet with the first oligomerization zone 300 inlet.

In one embodiment, as shown by FIG. 1, the present invention is a process for converting propane to distillate. The process includes passing a propane rich stream 20 to a dehydrogenation zone 100 to generate a first process stream 102 comprising propane and propylene. The first process stream 102 is passed to a first oligomerization zone 300, operated at a first set of reaction conditions, to generate a second process stream 302 comprising C₆+ olefins. The second process stream 302 is passed to a first fractionation unit 400 to generate a third stream comprising propane 402, and a fourth stream 404 comprising C₆+ hydrocarbons. The fourth stream 404 is passed to a second oligomerization zone 500, operated at a second set of reaction conditions, to generate a fifth process stream 502 comprising higher molecular weight hydrocarbons than the fourth stream 404. The fifth process stream 502 is passed to a second fractionation unit 600 to generate an olefinic process stream 602 and a sixth stream 604 comprising distillate range olefins. The sixth stream 604 is passed to a first complete saturation unit 700 to generate a seventh stream 702 having a reduced olefin content. The seventh stream 702 is passed to a stripper 800 to generate an overhead stream 802 comprising light ends, and a stripper bottoms stream 804 comprising distillate product.

The process can further include passing a portion, or all, of the third stream 402 to the dehydrogenation zone 100. The dehydrogenation process generates a process stream with a substantial amount of propane unconverted. The process in one aspect recycles the third stream to the dehydrogenation zone and comprises at least 50% of the feed to the dehydrogenation zone. The feed to the dehydrogenation zone preferably comprises a propane rich stream with at least 90% propane in the feed to the dehydrogenation zone.

The process can further include passing the first process stream 102 to a deethanizer 200 to generate an overhead stream 202 comprising C2 and lighter components, and a bottoms stream 204 comprising propane and propylene; and passing the bottoms stream 204 to the first oligomerization zone 300.

The process includes optional recycling of streams due to controlling the amount of oligomerization of the hydrocarbons. One aspect of the process includes passing a portion of the fifth process stream 502 to the second oligomerization zone 500, or recycling a portion of the second oligomerization zone 500 outlet to the inlet. Another aspect includes passing a portion of the distillate product stream 804 to the second oligomerization zone 500. Another aspect includes passing a portion of the distillate product stream 804 to the first oligomerization zone 300. And yet another aspect includes passing a portion of the fourth process stream 404 to the first oligomerization zone 300.

The process includes operation of the first oligomerization zone 300 at oligomerization reaction conditions, wherein the oligomerization reaction conditions include a reaction temperature between 100° C. to 250° C., and a reaction pressure between 100 kPa to 2000 kPa (absolute).

Another embodiment, as exhibited by FIG. 2, is a process for the conversion of propane to distillate. The process includes passing a propane rich stream 20 to a dehydrogenation reactor 100 to generate a first process stream 102 comprising propane and propylene. The first process stream 102 is passed to a deethanizer 200 to generate an overhead stream 202 comprising C2 and lighter components, and a bottoms stream 204 comprising propane and propylene. The bottoms stream 204 is passed to the first oligomerization zone 300, operated at a first set of reaction conditions, to generate a second process stream 302 comprising C₆+ olefins, The second process stream 302 is passed to a depropanizer 400 to generate a third stream 402 comprising propane, and a fourth stream 404 comprising C₆+ hydrocarbons. The fourth stream 404 is passed to a second oligomerization zone 500, operated at a second set of reaction conditions, to generate a fifth process stream 502 comprising distillate and olefins of higher molecular weight than the fourth stream 402. The fifth process stream 502 is passed to a complete saturation unit 700 to generate a process stream 702 comprising distillate and gasoline range hydrocarbons with reduced olefin content. The process stream 702 is passed to a distillate splitter 600 to generate a light end process stream 602 and a sixth stream 604 comprising distillate. The light end process stream 602, comprising gasoline range hydrocarbons and light ends, can be passed to a light end stripper 650 to remove light hydrocarbons generated by the complete saturation unit 700.

One aspect of this embodiment includes passing the third stream 402 to the dehydrogenation reactor 100. Another aspect includes passing the propane rich stream 20 to a contaminant removal zone 950 to generate the propane rich stream with reduced contaminants 22, and passing the reduced contaminants propane rich stream 22 to the dehydrogenation reactor 100.

The process can include passing a portion of the distillate process stream 702 with reduced olefin content or a portion of the sixth stream 604 comprising distillate to either, or both, the first oligomerization zone 300, or the second oligomerization zone 500.

Oligomerization conditions include the presence of a catalyst and the use of elevated temperatures and pressures. The specific temperature and pressure conditions used will depend, at least in part, upon the type of catalyst employed. Both homogeneous and heterogeneous catalysts can be used. Examples of homogeneous catalysts include hydrogen fluoride, boron trifluoride, and trifluoroacetic acid. Heterogeneous catalysts include suitable silica-aluminas, sulfated zirconias, and molecular sieves and supported metal-containing catalysts that often contain at least one element selected from Groups 3, 4, 8 to 10, and 14 of the Periodic Table. References herein to the Periodic Table are to the new IUPAC notation as shown on the Periodic Table of Elements in the inside front cover of the CRC Handbook of Chemistry and Physics, 80th Edition, 1999-2000, CRC Press, Boca Raton, Fla., USA.

In an exemplary embodiment, temperatures for the oligomerization are generally in the range of about 100° C. (212° F.) to 250° C. (482° F.), preferably 120° C. (248° F.) to 200° C. (392° F.), and pressures of from about 100 kPa(a) (14.5 psi(a)) to 2000 kPa(a) (290 psi(a)), preferably from about 110 kPa(a) (16 psi(a)) to 1000 kPa(a) (145 psi(a)). While oligomerization may be the predominant reaction in the oligomerization reactor2, some metathesis or dimerization may also occur.

### SPECIFIC EMBODIMENTS

While the following is described in conjunction with specific embodiments, it will be understood that this description is intended to illustrate and not limit the scope of the preceding description and the appended claims.

A first embodiment of the invention is an apparatus for the production of distillate, comprising a dehydrogenation zone having an inlet and an outlet for passage of an olefin enriched stream; a deethanizer unit having an inlet in fluid communication with the dehydrogenation zone outlet, an overhead outlet for light ends, and a bottoms outlet for C3s; a first oligomerization zone having an inlet in fluid communication with the dehydrogenation zone outlet, and an outlet for the passage of a hydrocarbon stream comprising C6 to C12 hydrocarbons; a first fractionation unit having an inlet in fluid communication with the first oligomerization zone outlet, and a first fractionation unit overhead outlet for the passage of C3s, and a first fractionation unit bottoms outlet for the passage of C₆+ hydrocarbons; a second oligomerization zone having an inlet in fluid communication with the first fractionation unit bottoms outlet, and an outlet; and a second fractionation unit having an inlet in fluid communication with the second oligomerization zone outlet, a second fractionation unit overhead outlet for the passage of an olefinic process stream, and a second fractionation unit bottoms outlet for the passage of a distillate process stream. An embodiment of the invention is one, any or all of prior embodiments in this paragraph up through the first embodiment in this paragraph further comprising a first conduit having an inlet connecting a portion of the second oligomerization zone outlet, and an outlet in fluid communication with the second oligomerization zone inlet. An embodiment of the invention is one, any or all of prior embodiments in this paragraph up through the first embodiment in this paragraph further comprising a first complete saturation process unit having an inlet in fluid communication with the second fractionation unit bottoms outlet and an outlet for the passage of a hydrogenated distillate process stream; and a product stripper having an inlet in fluid communication with the complete saturation unit outlet, a stripper overhead outlet for light ends, and a stripper bottoms outlet for distillate product. An embodiment of the invention is one, any or all of prior embodiments in this paragraph up through the first embodiment in this paragraph further comprising a second conduit having an inlet connecting a portion of the first fractionation unit overhead outlet, and an outlet in fluid communication with the inlet of the dehydrogenation unit. An embodiment of the invention is one, any or all of prior embodiments in this paragraph up through the first embodiment in this paragraph further comprising a second complete saturation process unit having an inlet in fluid communication with the second oligomerization zone outlet, and an outlet in fluid communication with the inlet to the second fractionation unit. An embodiment of the invention is one, any or all of prior embodiments in this paragraph up through the first embodiment in this paragraph further comprising a third conduit having an inlet in fluid communication with of the first fractionation unit bottoms stream, and an outlet in fluid communication with the inlet to the first oligomerization zone. An embodiment of the invention is one, any or all of prior embodiments in this paragraph up through the first embodiment in this paragraph further comprising a fourth conduit having an inlet in fluid communication with the product stripper bottoms outlet, and an inlet in fluid communication with the inlet to the second oligomerization zone. An embodiment of the invention is one, any or all of prior embodiments in this paragraph up through the first embodiment in this paragraph wherein the first oligomerization zone comprises a plurality of reactors in a series arrangement. An embodiment of the invention is one, any or all of prior embodiments in this paragraph up through the first embodiment in this paragraph wherein the first oligomerization zone includes a catalyst selected from the group consisting of zeolites having TON, MTT, MFI, MEL, AFO, AEL, EUO and FER type structures, solid phosphoric acid catalysts, and mixtures thereof. An embodiment of the invention is one, any or all of prior embodiments in this paragraph up through the first embodiment in this paragraph further comprising an aromatics removal unit having an inlet in fluid communication with the deethanizer bottoms outlet, and an outlet in fluid communication with the first oligomerization zone inlet. An embodiment of the invention is one, any or all of prior embodiments in this paragraph up through the first embodiment in this paragraph further comprising a fifth conduit having an inlet connecting a portion of the second oligomerization zone outlet, and an outlet in fluid communication with the first oligomerization zone inlet. An embodiment of the invention is one, any or all of prior embodiments in this paragraph up through the first embodiment in this paragraph further comprising a sixth conduit having an inlet in fluid communication with the product stripper bottoms outlet, and an inlet in fluid communication with the inlet to the first oligomerization zone.

A second embodiment of the invention is an apparatus for the production of distillate, comprising a dehydrogenation unit having an inlet and an outlet for passage of an olefin enriched stream; a deethanizer unit having an inlet in fluid communication with the dehydrogenation unit outlet, an overhead outlet for light ends, and a bottoms outlet for C3s; an aromatics removal unit having an inlet in fluid communication with the deethanizer bottoms outlet, and an outlet for a C3 stream with reduced aromatics; a first oligomerization zone having an inlet in fluid communication with the aromatics removal unit outlet, and an outlet for the passage of a hydrocarbon stream comprising C6 to C12 hydrocarbons; a first fractionation unit having an inlet in fluid communication with the first oligomerization zone outlet, and a first fractionation unit overhead outlet for the passage of C3s, and a first fractionation unit bottoms outlet for the passage of C6 to C12 hydrocarbons; a second oligomerization zone having an inlet in fluid communication with the first fractionation unit bottoms outlet, and an outlet; a second fractionation unit having an inlet in fluid communication with the second oligomerization zone outlet, a second fractionation unit overhead outlet for the passage of an olefinic process stream, and a second fractionation unit bottoms outlet for the passage of a distillate process stream; and a first conduit in direct communication with the outlet of the first fractionation overhead and the inlet to the dehydrogenation unit. An embodiment of the invention is one, any or all of prior embodiments in this paragraph up through the second embodiment in this paragraph further comprising a complete saturation process unit having an inlet in fluid communication with the second oligomerization unit outlet and an outlet in fluid communication with the second fractionation unit. An embodiment of the invention is one, any or all of prior embodiments in this paragraph up through the second embodiment in this paragraph wherein the first oligomerization zone includes a catalyst selected from the group consisting of zeolites having TON, MTT, MFI, MEL, AFO, AEL, EUO and FER type structures, solid phosphoric acid, and mixtures thereof. An embodiment of the invention is one, any or all of prior embodiments in this paragraph up through the second embodiment in this paragraph further comprising a first conduit having an inlet in fluid communication with the first fractionation unit overhead outlet, and an outlet in fluid communication with the dehydrogenation unit inlet. An embodiment of the invention is one, any or all of prior embodiments in this paragraph up through the second embodiment in this paragraph further comprising a second conduit having an inlet in fluid communication with the complete saturation process unit outlet, and an inlet in fluid communication with the second oligomerization zone inlet. An embodiment of the invention is one, any or all of prior embodiments in this paragraph up through the second embodiment in this paragraph wherein the dehydrogenation unit is a propane dehydrogenation unit. An embodiment of the invention is one, any or all of prior embodiments in this paragraph up through the second embodiment in this paragraph wherein the first oligomerization zone and the second oligomerization zone each comprise a plurality of reactors in a series arrangement. An embodiment of the invention is one, any or all of prior embodiments in this paragraph up through the second embodiment in this paragraph further comprising a contaminant removal zone having an inlet to a feedstream and an outlet in fluid communication with the dehydrogenation unit inlet.

A third embodiment of the invention is a process for converting propane to distillate, comprising passing a propane rich stream to a dehydrogenation zone to generate a first process stream comprising propane and propylene; passing the first process stream to a first oligomerization zone, operated at a first set of reaction conditions, to generate a second process stream comprising C₆+ olefins; passing the second process stream to a first fractionation unit to generate a third stream comprising propane, and a fourth stream comprising C₆+ hydrocarbons; passing the fourth stream to a second oligomerization zone, operated at a second set of reaction conditions, to generate a fifth process stream comprising higher molecular weight hydrocarbons than the fourth stream; passing the fifth process stream to a second fractionation unit to generate an olefinic process stream and a sixth stream comprising distillate range olefins; passing the sixth stream to a first complete saturation unit to generate a seventh stream having reduced olefin content; and passing the seventh stream to a stripper to generate an overhead stream comprising light ends, and a stripper bottoms stream comprising distillate product. An embodiment of the invention is one, any or all of prior embodiments in this paragraph up through the third embodiment in this paragraph wherein the first oligomerization zone comprises a plurality of reactors in series. An embodiment of the invention is one, any or all of prior embodiments in this paragraph up through the third embodiment in this paragraph further comprising passing a portion of the third stream to the dehydrogenation zone. An embodiment of the invention is one, any or all of prior embodiments in this paragraph up through the third embodiment in this paragraph wherein the third stream fed to the dehydrogenation zone comprises at least 50% of the feed to the dehydrogenation zone. An embodiment of the invention is one, any or all of prior embodiments in this paragraph up through the third embodiment in this paragraph wherein the propane rich stream comprises at least 90% propane. An embodiment of the invention is one, any or all of prior embodiments in this paragraph up through the third embodiment in this paragraph further comprising passing the first process stream to a deethanizer to generate an overhead stream comprising C2 and lighter components, and a bottoms stream comprising propane and propylene; and passing the bottoms stream to the first oligomerization zone. An embodiment of the invention is one, any or all of prior embodiments in this paragraph up through the third embodiment in this paragraph further comprising passing a portion of the fifth process stream to the second oligomerization zone. An embodiment of the invention is one, any or all of prior embodiments in this paragraph up through the third embodiment in this paragraph further comprising passing a portion of the distillate product to the second oligomerization zone. An embodiment of the invention is one, any or all of prior embodiments in this paragraph up through the third embodiment in this paragraph further comprising passing a portion of the distillate product to the first oligomerization zone. An embodiment of the invention is one, any or all of prior embodiments in this paragraph up through the third embodiment in this paragraph further comprising passing a portion of the fourth process stream to the first oligomerization zone. An embodiment of the invention is one, any or all of prior embodiments in this paragraph up through the third embodiment in this paragraph wherein the first oligomerization zone includes a catalyst selected from the group consisting of zeolites having TON, MTT, MFI, MEL, AFO, AEL, EUO and FER type structures, solid phosphoric acid, and mixtures thereof. An embodiment of the invention is one, any or all of prior embodiments in this paragraph up through the third embodiment in this paragraph wherein the second oligomerization zone includes a catalyst selected from the group consisting of zeolites having TON, MTT, MFI, MEL, AFO, AEL, EUO and FER type structures, solid phosphoric acid, and mixtures thereof. An embodiment of the invention is one, any or all of prior embodiments in this paragraph up through the third embodiment in this paragraph wherein the first set of reaction conditions and the second set of reaction conditions includes a reaction temperature between 100° C. to 250° C. An embodiment of the invention is one, any or all of prior embodiments in this paragraph up through the third embodiment in this paragraph wherein the first set of reaction conditions and the second set of reaction conditions includes a reaction pressure between 100 kPa to 2000 kPa (absolute).

A fourth embodiment of the invention is a process for converting propane to distillate, comprising passing a propane rich stream to a dehydrogenation reactor to generate a first process stream comprising propane and propylene; passing the first process stream to a deethanizer to generate an overhead stream comprising C2 and lighter components, and a bottoms stream comprising propane and propylene; passing the bottoms stream to the first oligomerization zone, operated at a first set of reaction conditions, to generate a second process stream comprising C₆+ olefins; passing the second process stream to a depropanizer to generate a third stream comprising propane, and a fourth stream comprising C₆+ hydrocarbons; passing the fourth stream to a second oligomerization zone, operated at a second set of reaction conditions, to generate a fifth process stream comprising distillate and olefins of higher molecular weight than the fourth stream; passing the fifth process stream to a distillate splitter to generate an olefinic process stream and a sixth stream comprising distillate; passing the sixth stream to a first complete saturation unit to generate a seventh stream having reduced olefin content; and passing the seventh stream to a light ends stripper to generate an overhead stream comprising light ends, and a light ends stripper bottoms stream comprising distillate product. An embodiment of the invention is one, any or all of prior embodiments in this paragraph up through the fourth embodiment in this paragraph wherein the first set or reaction conditions includes contacting with a catalyst selected from the group consisting of zeolites having TON, MTT, MFI, MEL, AFO, AEL, EUO and FER type structures, solid phosphoric acid, and mixtures thereof An embodiment of the invention is one, any or all of prior embodiments in this paragraph up through the fourth embodiment in this paragraph wherein the second set or reaction conditions includes contacting with a catalyst selected from the group consisting of zeolites having TON, MTT, MFI, MEL, AFO, AEL, EUO and FER type structures, solid phosphoric acid, and mixtures thereof. An embodiment of the invention is one, any or all of prior embodiments in this paragraph up through the fourth embodiment in this paragraph further comprising passing the third stream to the dehydrogenation reactor. An embodiment of the invention is one, any or all of prior embodiments in this paragraph up through the fourth embodiment in this paragraph further comprising passing the propane rich stream to a contaminant removal zone to generate the propane rich stream with reduced contaminants, and passing the reduced contaminants propane rich stream to the dehydrogenation reactor. An embodiment of the invention is one, any or all of prior embodiments in this paragraph up through the fourth embodiment in this paragraph further comprising passing a portion of the distillate product to the first oligomerization zone.

Without further elaboration, it is believed that using the preceding description that one skilled in the art can utilize the present invention to its fullest extent and easily ascertain the essential characteristics of this invention, without departing from the spirit and scope thereof, to make various changes and modifications of the invention and to adapt it to various usages and conditions. The preceding preferred specific embodiments are, therefore, to be construed as merely illustrative, and not limiting the remainder of the disclosure in any way whatsoever, and that it is intended to cover various modifications and equivalent arrangements included within the scope of the appended claims.

In the foregoing, all temperatures are set forth in degrees Celsius and, all parts and percentages are by weight, unless otherwise indicated.

What is claimed is:

1. An apparatus for the production of distillate, comprising:
a dehydrogenation zone having an inlet and an outlet for passage of an olefin enriched stream;
a deethanizer unit having an inlet in fluid communication with the dehydrogenation zone outlet, an overhead outlet for light ends, and a bottoms outlet for C3s;
a first oligomerization zone having an inlet in fluid communication with the dehydrogenation zone outlet, and an outlet for the passage of a hydrocarbon stream comprising C6 to C12 hydrocarbons;
a first fractionation unit having an inlet in fluid communication with the first oligomerization zone outlet, and a first fractionation unit overhead outlet for the passage of C3s, and a first fractionation unit bottoms outlet for the passage of C₆+ hydrocarbons;
a second oligomerization zone having an inlet in fluid communication with the first fractionation unit bottoms outlet, and an outlet; and
a second fractionation unit having an inlet in fluid communication with the second oligomerization zone outlet, a second fractionation unit overhead outlet for the passage of an olefinic process stream, and a second fractionation unit bottoms outlet for the passage of a distillate process stream.

2. The apparatus of claim 1 further comprising a first conduit having an inlet connecting a portion of the second oligomerization zone outlet, and an outlet in fluid communication with the second oligomerization zone inlet.

3. The apparatus of claim 1 further comprising:
a first complete saturation process unit having an inlet in fluid communication with the second fractionation unit bottoms outlet and an outlet for the passage of a hydrogenated distillate process stream; and
a product stripper having an inlet in fluid communication with the complete saturation unit outlet, a stripper overhead outlet for light ends, and a stripper bottoms outlet for distillate product.

4. The apparatus of claim 1 further comprising a second conduit having an inlet connecting a portion of the first fractionation unit overhead outlet, and an outlet in fluid communication with the inlet of the dehydrogenation unit.

5. The apparatus of claim 1 further comprising:
a second complete saturation process unit having an inlet in fluid communication with the second oligomerization zone outlet, and an outlet in fluid communication with the inlet to the second fractionation unit.

6. The apparatus of claim 1 further comprising a third conduit having an inlet in fluid communication with of the first fractionation unit bottoms stream, and an outlet in fluid communication with the inlet to the first oligomerization zone.

7. The apparatus of claim 3 further comprising a fourth conduit having an inlet in fluid communication with the product stripper bottoms outlet, and an inlet in fluid communication with the inlet to the second oligomerization zone.

8. The apparatus of claim 1 wherein the first oligomerization zone comprises a plurality of reactors in a series arrangement.

9. The apparatus of claim 1 wherein the first oligomerization zone includes a catalyst selected from the group consisting of zeolites having TON, MTT, MFI, MEL, AFO, AEL, EUO and FER type structures, solid phosphoric acid catalysts, and mixtures thereof.

10. The apparatus of claim 1 further comprising an aromatics removal unit having an inlet in fluid communication with the deethanizer bottoms outlet, and an outlet in fluid communication with the first oligomerization zone inlet.

11. The apparatus of claim 1 further comprising a fifth conduit having an inlet connecting a portion of the second oligomerization zone outlet, and an outlet in fluid communication with the first oligomerization zone inlet.

12. The apparatus of claim 3 further comprising a sixth conduit having an inlet in fluid communication with the product stripper bottoms outlet, and an inlet in fluid communication with the inlet to the first oligomerization zone.

13. An apparatus for the production of distillate, comprising:
a dehydrogenation unit having an inlet and an outlet for passage of an olefin enriched stream;
a deethanizer unit having an inlet in fluid communication with the dehydrogenation unit outlet, an overhead outlet for light ends, and a bottoms outlet for C3s;
an aromatics removal unit having an inlet in fluid communication with the deethanizer bottoms outlet, and an outlet for a C3 stream with reduced aromatics;
a first oligomerization zone having an inlet in fluid communication with the aromatics removal unit outlet, and an outlet for the passage of a hydrocarbon stream comprising C6 to C12 hydrocarbons;
a first fractionation unit having an inlet in fluid communication with the first oligomerization zone outlet, and a first fractionation unit overhead outlet for the passage of C3s, and a first fractionation unit bottoms outlet for the passage of C6 to C12 hydrocarbons;
a second oligomerization zone having an inlet in fluid communication with the first fractionation unit bottoms outlet, and an outlet;
a second fractionation unit having an inlet in fluid communication with the second oligomerization zone outlet, a second fractionation unit overhead outlet for the passage of an olefinic process stream, and a second fractionation unit bottoms outlet for the passage of a distillate process stream; and
a first conduit in direct communication with the outlet of the first fractionation overhead and the inlet to the dehydrogenation unit.

14. The apparatus of claim 13 further comprising a complete saturation process unit having an inlet in fluid communication with the second oligomerization unit outlet and an outlet in fluid communication with the second fractionation unit.

15. The apparatus of claim 13 wherein the first oligomerization zone includes a catalyst selected from the group consisting of zeolites having TON, MTT, MFI, MEL, AFO, AEL, EUO and FER type structures, solid phosphoric acid, and mixtures thereof.

16. The apparatus of claim 13 further comprising a first conduit having an inlet in fluid communication with the first fractionation unit overhead outlet, and an outlet in fluid communication with the dehydrogenation unit inlet.

17. The apparatus of claim 14 further comprising a second conduit having an inlet in fluid communication with the complete saturation process unit outlet, and an inlet in fluid communication with the second oligomerization zone inlet.

18. The apparatus of claim 13 wherein the dehydrogenation unit is a propane dehydrogenation unit.

19. The apparatus of claim 13 wherein the first oligomerization zone and the second oligomerization zone each comprise a plurality of reactors in a series arrangement.

20. The apparatus of claim 13 further comprising a contaminant removal zone having an inlet to a feedstream and an outlet in fluid communication with the dehydrogenation unit inlet.
